(19) United States
(12) Patent Application Publication
(10) Pub. No.: US 2003/0073974 A1
(43) Pub. Date: Apr. 17, 2003

(54) Bowel irrigation probe

(76) Inventor: Malcolm Ian Falconer, London (GB)

Correspondence Address: JACOBSON HOLMAN PLLC, 400 SEVENTH STREET N.W., SUITE 600, WASHINGTON, DC, 20004, US

(21) Appl. No.: 10/058,346

(22) Filed: Jan. 30, 2002

(30) Foreign Application Priority Data
Oct. 12, 2001 (DK) PA 2001 01511

(51) Int. Cl. A61M 5/178

(52) U.S. Cl. 604/514; 606/197

(57) ABSTRACT

A probe for use with a bowel irrigation system that is suitable for use by persons suffering from limited limb movement. The probe includes a shaft with a liquid supply duct passing therethrough for supplying irrigation liquid from a proximal end of the shaft to an outlet on the distal end thereof. A flexible element having a liquid absorbent material is positioned near the outlet and expands upon contact with liquid to ensure retention of the probe when in use.

### FIELD OF INVENTION

This invention relates to a probe for use with a bowel irrigation system and more particularly, but not exclusively, to a probe which can be easily used by disabled persons suffering from limited limb movement, for instance due to a spinal cord injury or being spina bifida or MS sufferers.

### BACKGROUND OF INVENTION

In order to successfully carry out an anal irrigation procedure, a probe which is connected to a source of irrigation liquid needs to be inserted into the user's anal opening and retained therein until the required amount of liquid has been fed into their bowel. In prior art probes, locating the probe in the anal opening is usually achieved by means of a balloon on the probe which can be inflated once it has been inserted in the anal opening. However, the pressure applied to rectum while the balloon is being inflated can sometimes set off a reflex action in the bowel which causes it to empty prematurely and additionally eject the probe. This is most unsatisfactory and distressing for the user as the evacuated contents of the bowel have to be cleared up and the procedure repeated.

It is therefore an object of the invention to provide a probe with expandable means on it which is easy and simple to use and which overcome or substantially reduce the above mentioned problems.

According to the invention, there is provided a probe comprising a shaft having proximal and distal ends, the shaft having a liquid supply duct therethrough for supplying irrigation liquid to an outlet adjacent said distal end, and expandable means on the shaft adjacent said outlet which are operable, in use, to retain the probe in the user's anal opening and make a fluid tight seal therewith, the invention being characterised in that the expandable means comprises a flexible expandable enclosure with an absorbent material therein which expands when contacted with a liquid. In connection with this invention, the distal end of the probe is understood to be the insertable end of the probe.

Preferably the shaft has an outlet therein to allow irrigation liquid fed therethrough to flow into contact with the expandable material in the flexible enclosure.

In a preferred embodiment, the expandable enclosure is water impermeable and comprises two discrete chambers separated by a releasable seal. The enclosure can however be water permeable. Conveniently, release means are connected to the seal which are operable to break the seal and allow the two discrete chambers to become a single larger chamber into which the expanded liquid absorbent material can disperse to allow the enclosure to be collapsed for removal of the probe from the user's anal opening. In the preferred embodiment, the release means is a pull string and the releasable seal is a rupturable weld attaching an annular region of the enclosure to the shaft intermediate the opposite ends of said enclosure

Alternatively, rupture means can be provided on the enclosure which are operable to puncture the enclosure and allow the expanded contents thereof to disperse to the exterior of the enclosure and allowing it to collapse.

The expandable enclosure is preferably a stretchable balloon. It could however be a non-stretchable balloon of the required expanded capacity, said balloon being collapsed prior to insertion of the probe in the user's anal opening.

It is an advantage of the probe of the invention that the expandable cuff can be increased in size to make the required seal with the user's anal opening without the need for the user to carry out any additional step other than opening the supply of the irrigation liquid to the probe. It is therefore extremely easy and convenient to use.

### BRIEF DESCRIPTION OF THE DRAWINGS

The invention will now be described, by way of example only, with reference to the accompanying drawings, in which:

FIG. 1 is a perspective view of a portable bowel irrigation system incorporating a probe of the invention;

FIG. 2 is a schematic view showing the system of FIG. 1 in use;

FIG. 3 is a side view of a probe for use with the system shown in FIGS. 1 and 2 with the balloon in its normal collapsed condition;

FIG. 4 is a side view of the probe shown in FIG. 3 but with the balloon in its expanded condition; and

FIG. 5 is an enlarged schematic cross section of the probe shown in FIG. 3.

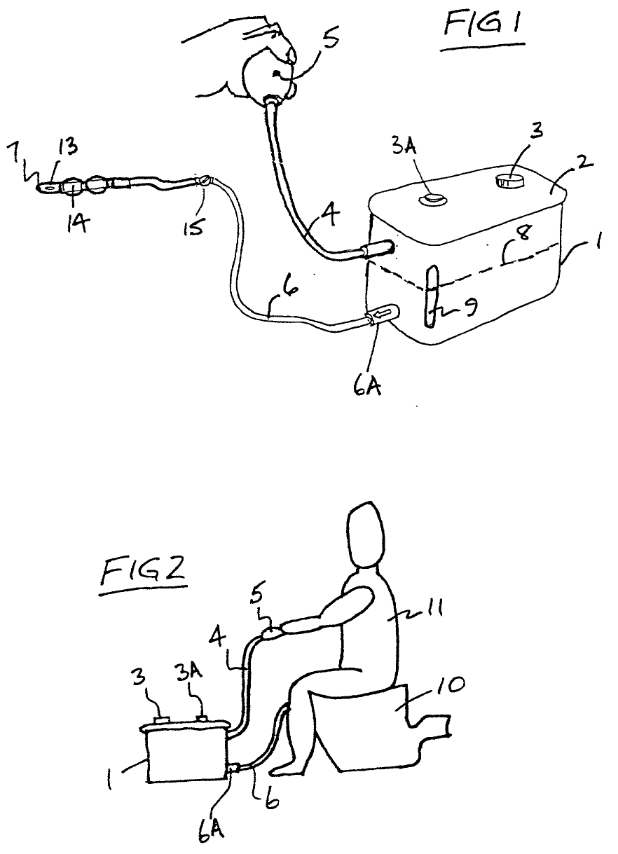

Referring now to the drawings, there is shown in FIG. 1 a bowel irrigation system which comprises a free-standing reservoir 1 for irrigation liquid with a top 2 fitted thereto having an inlet therein closed by a screw cap 3. The top 2 makes an airtight seal with the reservoir 1 to provide a sealed container for the irrigation liquid. A pressure relief valve 3A is mounted on the lid 2 for reasons to be explained hereafter. The sidewall of the reservoir 1 can have an optical sight glass 9 in it to indicate the level of liquid inside the reservoir 1.

A flexible tube 4 is connected to the sidewall of the reservoir 1 and the outlet therefrom opens into the interior of the reservoir 1 above the upper level 8 of the liquid therein. A compressible member in the form of a rubber bulb 5 is attached to the end of the tube 4.

A second tube 6 is also connected at one end to the reservoir 1 via a one-way valve 6A which opens into the bottom thereof below the liquid level 8. A probe 7 is attached to the free end of tube 6. A tap or clamp 15 is fitted in the tube 6 intermediate its ends. The probe 7, which preferably is provided having a relatively small diameter to ease insertion of the probe into the body opening, has an outlet 13 for irrigation liquid and an expandable cuff 14 which will be described in more detail hereafter with reference to FIGS. 3-5.

The way in which the bowel irrigation system shown in FIG. 1 is used by a user, carer or helper will now be described with reference to FIG. 2.

As can be seen from the drawing, user 11 must first sit on toilet 10. If the user is in a wheelchair, it will be appreciated that this should not be too difficult as they can slide from the wheelchair onto the toilet. The liquid reservoir is placed on the floor opposite the user and the user 11 can then insert the tube 6 with probe 7 at its distal end into position in their anal opening. The user now repeatedly squeezes the bulb 5 to pressurise the container 1 and thereby displace the liquid through the tube 6 to the probe 7 where it exits via outlet 13 into the user's colon. So long as the user continues to squeeze the bulb 5, the irrigation liquid will continue to flow. Pressure 3A prevents over-pressurisation of the container 1 and thus the pressure of the liquid entering the user's bowel. The valve 3A is set to vent at approximately 2.5 m of water. The valve 3A can be a simple pressure relief valve of known type which releases at the pre-set pressure or it could be a simple stopper which pops out of the lid at the required predetermined pressure. Initial pumping of the water through the probe 7 encloses the cuff 14 which locates it in the user's anal opening and makes a seal therewith.

FIGS. 3-5 of the drawings show in more detail the probe 7 of the invention which has just been described briefly with reference to FIGS. 1 and 2. The probe 7 comprises a hollow shaft or tube 7A made of a moulded plastics material such as polyethylene, pvc or polyurethane having an outlet 13 adjacent its distal end for irrigation liquid supplied to it from the reservoir 1 (not shown). The expandable cuff 14 comprises two individual cuffs 14A and 14B which are sealing secured in known manner e.g. by welding at 16A, 16B and 16C to the shaft 7 to provide first and second chambers 18 and 19 (see FIG. 5). The cuff 14 is preferably made of a bicomponent non woven material made from polyethylene fibres with a polypropylene core. Other non woven materials e.g. PU could be used. Elastic impermeable materials such as PU or latex could also be used. Holes 21 in the wall of the shaft 7A open into the first chamber 18 which is filled with a liquid absorbent material 20 which expands when it comes into contact with liquid.

A preferred liquid absorbent material 20 is sodium polyacrylate. It can be provided in different ways e.g. such as in a powder form or laminated into a sheet between papers. Alternative absorbents can be chosen from a group comprising inorganic materials, such as liquid gels, or organic compounds, such as cross linked polymers, or alginates, reticular carboxymethylcelluloses, grafted starches, natural modified polysaccharides or synthetic derivatives of acrylamides, acrylonitriles or polyacrylates.

A pull string 22 is attached at 23 to the weld 16B. The proximal end of the tube 7A has a series of axially spaced annular ribs 17 on it onto which the tube 6 can be fitted.

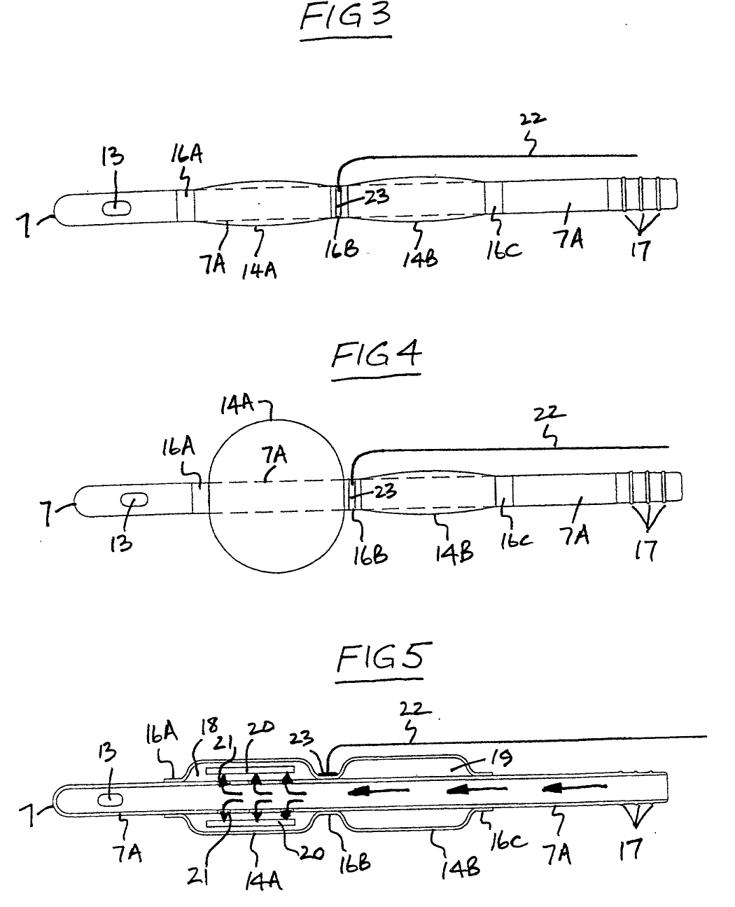

The probe 7 operates in the following manner. When irrigation liquid is supplied to the probe from the reservoir 1, it flows through the interior of the shaft 7A as indicated by the arrows and passes through apertures 21 into the first chamber 18 where it contacts the super absorbent material 20 which swells and expands to a diameter of e.g. approximately 60 mm to enlarge the cuff 14A to its configuration shown in FIG. 4. Expansion of the absorbent, and thereby of the cuff, will occur concurrently with expansion of the diameter of rectum during administration of the irrigation liquid thereby constantly providing a liquid tight sealing between the cuff and the wall of rectum.

In order to avoid diffusion of superabsorbent material into the cavity of the shaft 7A a layer of liquid permeable material (not shown) may present covering holes 21.

It will be understood that the cuff may be constructed to reach a diameter of appropriate size, be it larger or smaller than the suggested 60 mm depending of the size needed to seal the relevant body opening of a specific user.

Once the cuff 14A is fully expanded, the probe 7 makes a seal with the user's anal opening and should not be intendedly removed therefrom until the cuff 14 is collapsed. Because the liquid absorbent material 20 expands gradually when it comes into contact with the irrigation liquid, it exerts very little pressure on the user's colon so there is little or no risk of it stimulating a reflex action which could cause the bowel to accidentally or prematurely evacuate its contents.

Irrigation water continues to be pumped into the user's bowel through the probe 7 via outlet 13 until the user is ready to empty their bowel. To do this, they pull on the string 22 which ruptures the weld 16B and pulls it away from the shaft 7A thereby opening a passageway between the first and second chambers 18 and 19. Because of the elasticity or resilience of the stretched cuff 14A, it applies a radially inwardly directed force on the expanded liquid absorbent material 20 which is now in a gel form so it can disperse axially of the probe into the second chamber 19 thereby allowing the first cuff 14A to contract back to its original configuration shown in FIG. 3. The user can then easily withdraw the probe from their anal opening and evacuate the contents of their bowel into an appropriately placed container (not shown).

Instead of the pull string 22 being used to rupture the weld 16B, the weld could be made to rupture automatically when the pressure in the first chamber 18 reaches a predetermined level. Alternatively, seal 16B could be created using a noose on the end of the string 22 which releases when the string 22 is pulled. As a further alternative, the seal 16B could incorporate a frangible tear line which opens when the string 22 attached thereto is pulled by the user.

Instead of providing a tearing force to rupture the seal between the two chambers of the cuff 14A and 14B, a force can be exerted on a cuff-sealing member encircling the probe between the two chambers thereby causing the sealing member to slide coaxially along the probe and allowing the volume of the absorbent filled chamber to increase. This will allow for the swelled superabsorbent material now in the form of a gel to disperse axially, thereby allowing the first chamber to contract back to its original configuration shown in FIG. 3. The user can then easily withdraw the probe.

As a further alternative, a tear strip could be incorporated into the wall of the cuff 14A with a string 22 connected to it so that when the string is pulled, the wall of the cuff 14A is punctured and the contents of the first chamber 18 can exit from the first cuff 14A into the user's bowel thereby allowing the cuff 14A to contract to its original configuration shown in FIG. 3.

As illustrated in FIG. 5, the superabsorbent material can be present in the cuff-member 14A situated adjacent the outlet 13 of the probe 7. In an alternative embodiment (not shown) the superabsorbent material is provided in the cuff-member 14B and in stead of apertures 21 the probe is provided with apertures situated in relation to cuff member 14B thereby enabling liquid communication with the superabsorbent for swelling thereof. Before removing the probe from the body opening the cuff member, axially dispersion of the formed gel is enabled as described above. This dispersion can now even occur during withdrawal of the probe due to the force exerted on the cuff from the tissue or from the muscle of the body opening.

Although the probe has been described for use with the irrigation system shown in FIGS. 1 and 2, it is not specifically designed for use with such a system and can be used with any form of bowel irrigation system. It should also be noted that the probe is intended to be used only once and thereafter thrown away.

Although the irrigation system shown in FIGS. 1 and 2 and described above is primarily intended for use by a user without outside assistance, it is to be understood that the probe of the invention can be used with systems which require third party insertion of the probe into the patient or user or other outside assistance.

Although the invention has been described in relation to its use in a retrograde irrigation procedure, i.e. one in which the probe is inserted into the user's anal opening, it could be used in any irrigation procedure be it e.g. a stomal, ileostomal or an antegrade irrigation procedure in conjunction with a Malone irrigator whereby the probe is inserted into the exposed end of a stoma protruding through the user's stomach wall and connected to the user's bowel.

The irrigation medium to be used with the system of the invention can be any suitable medium e.g. tap water, isotonic salt water, sterile water or oily substances.

1. An probe comprising a shaft having proximal and distal ends, the shaft having a liquid supply duct therethrough for supplying irrigation liquid to an outlet adjacent said distal end, and expandable means on the shaft adjacent said outlet, characterised in that the expandable means comprises a flexible expandable enclosure (14) with a liquid absorbent material (20) therein which expands when contacted with a liquid.

2. A probe as claimed in claim 1 characterised in that the shaft (7A) has an outlet (21) therein to allow irrigation liquid fed therethrough to flow into contact with the expandable material (20) in the flexible enclosure (14).

3. A probe as claimed in claim 1 characterised in that the expandable enclosure (14) is made of a liquid impermeable material.

4. A probe as claimed in any preceding claim characterised in that rupture means (23) are provided on the expandable enclosure (14) which are operable to puncture the enclosure (14) and allow the contents (20) thereof to disperse to the exterior of the enclosure (14) and thereby collapse said enclosure.

5. A probe as claimed in any preceding claim characterised in that the expandable enclosure (14) comprises two discrete chambers (18,19) separated by a releasable seal (16B).

6. A probe as claimed in claim 5 characterised in that the releasable seal (16B) is a rupturable weld attaching an annular region of the enclosure (14) to the shaft (7A) intermediate the opposite ends thereof.

7. A probe as claimed in claim 5 or claim 6 characterised in that release means (22) are connected to the seal (16B) which are operable to break the seal and allow the two discrete chambers (18,19) to become a single larger chamber into which the expanded liquid absorbent material (20) can disperse to allow the enclosure (14) to be collapsed for removal of the probe (7) from the user's anal opening.

8. A probe as claimed in claim 7 wherein the release means is a pull string 22.

9. A probe as claimed in any preceding claim characterised in that the expandable enclosure (14) is a stretchable balloon.
